(19) United States
(12) Patent Application Publication
(10) Pub. No.: US 2024/0319156 A1
(43) Pub. Date: Sep. 26, 2024

(54) DEVICE FOR TESTING VAPORIZABLE FLUIDS IN A HUMAN AIRWAY MODEL

(71) Applicant: Rowan University, Glassboro, NJ (US)

(72) Inventors: Vincent Manna, Magnolia, NJ (US); Salvatore Caradonna, Voorhees, NJ (US)

(21) Appl. No.: 18/679,043

(22) Filed: May 30, 2024

Related U.S. Application Data
(63) Continuation of application No. PCT/US2023/023457, filed on May 25, 2023.
(60) Provisional application No. 63/347,219, filed on May 31, 2022.
(60) Provisional application No. 63/348,771, filed on Jun. 3, 2022.

(51) Int. Cl. G01N 33/00

(52) U.S. Cl. CPC G01N 33/0073

(57) ABSTRACT

A testing device for testing the impact of aerosolized compounds, such as e-cigarette vapors, metered-dose inhaled corticosteroids, and nebulized medications, on a human airway model system/tissue for purposes related to basic and clinical research, diagnostics, and personalized medicine. The device includes an intake fan assembly, a housing defining an exposure chamber, a holder supported on said housing for supporting an aerosol-generating device; and a control system operable to selectively energize the intake fan to draw the aerosolized compound into the exposure chamber, where human tissue is disposed. The testing device may also include an actuator operable by the control system to cause the aerosol-generating device to generate the aerosolized compound. The testing device may also include an exhaust fan operable by the control system to exhaust the aerosolized compound from the exposure chamber. Testing device components may be constructed of a biocompatible material that is autoclavable without detrimental degradation.

### CROSS-REFERENCE TO RELATED APPLICATIONS

This application is a continuation of International Application No. PCT/US2023/023457, filed May 25, 2023, which claims the benefit of priority, under 35 U.S.C. § 119(e), of U.S. provisional patent application Nos. 63/347,219, filed May 31, 2022, and 63/348,771, filed Jun. 3, 2022, the entire disclosures of each of which are hereby incorporated herein by reference.

### FIELD OF THE INVENTION

The present invention relates generally to analytical testing devices, and more particularly to a testing device for testing the impact of aerosolized compounds on a human airway model system.

### DISCUSSION OF RELATED ART

E-cigarettes, sometimes called “vape pens,” are portable battery-powered devices that are usable to provide a vaping experience somewhat similar to a tobacco-smoking experience, but without the use of tobacco. These devices are filled and refilled with a vaporizable “vape-liquid,” which is used to produce a vapor aerosol that is then inhaled by the user via the device. Generally speaking, such vape pens generally including a housing containing a battery and supporting a button/switch operable by a user to vaporize vape liquid housing in a cartridge supported on the vape pen, so that the vapor can be inhaled via a mouthpiece/tip of the device, as will be appreciated from FIG. 1.

By way of example, vapor is produced by passing a current through a wire wrapped around a wick soaked in vape-liquid, which then combusts to form the vapor that is inhaled. The vape liquids may be developed to include active ingredients such as nicotine and Tetrahydrocannabinol (THC) or Cannabidiol (CBD).

Vaping has become quite popular in recent years. It is estimated that from 2011 to 2018, vape-pen use in high school students increased from 1.5% to 20.8%. Additionally, the global vape industry has a market size that was valued at $12.41 billion in 2019, and is projected to increase by more than 20% over the following 5 years.

It has begun to be observed or believed that vaping has adverse health effects, including respiratory and gastrointestinal problems. The Center for Disease Control and Prevent (CDC) has introduced the term EVALI to described e-cigarette or vaping associated lung injuries/illnesses.

Accordingly, it may be desirable to test such vaping liquids, and even more desirable to test the impact of such vaping liquids on human tissues. Various smoking and/or vaping machines are available, for performing some manners of testing. Examples of such devices include a Transwell insert exposure chamber commercially-available from Cultex Technology, and a Smoking Machine commercially-available from Borgwaldt. These devices tend to be large/very large, heavy and/or expensive free-standing machines that include ventilation and/or fume control. More particularly, these smoking research devices are very large pieces of equipment that require considerable training for their operation and maintenance, in addition to being very limited in application. The smoking machine must be combined with the Cultex Transwell insert exposure chamber to utilize human airway model tissues for cigarette smoke research. The machine must be further modified to adapt for the usage of e-cigarettes and vape pens. There are no options to generate aerosols from other sources such as metered-dose inhalers or medical nebulizers.

What is needed is a testing device designed to be paired with human model airway tissues for testing the impact of aerosolized compounds, such as those generated from e-cigarette vape-liquids, metered-dose inhalers, and medical nebulizers, so that the safety, risks, and ramifications of these compounds can be assessed. Further, what is a needed is such a device that is sufficiently lightweight and compact to be readily usable within a conventional fume hood, so that device-specific ventilation and/or fume control structure and associated cost and complexity can be avoided, to the safety (or risks) of vaping and/or particular vape liquids can be assessed. Such a device has application in both basic and translational research settings that are modeling respiratory diseases, inflammation, inhaled chemotherapeutics, or respiratory drug delivery. There are clinical applications with the device by using it with patient-derived airway model tissue to design personalized medicine treatment plans for various respiratory illnesses with known inflammatory components, such as asthma or chronic obstructive pulmonary disease.

Human airway model tissue inside of such a device could be used to monitor ambient air for the presence of pathogenic viruses, bacteria, and yeast. This would be of utility in hospital settings that are especially concerned with rapidly identifying the presence of communicable diseases such as in intensive care units, neonatal intensive care units, infant nursery wards, surgical centers, chemotherapy centers, and organ transplant facilities.

### SUMMARY

The present invention relates generally to analytical testing devices, and more particularly to a testing device for testing the impact of aerosolized compounds, such as e-cigarette vapors, metered-dose inhaled corticosteroids, and nebulized medications, on a human airway model system for purposes related to basic and clinical research, diagnostics, and personalized medicine.

### BRIEF DESCRIPTION OF THE FIGURES

An understanding of the following description will be facilitated by reference to the attached drawings, in which:

FIG. 1 is an exploded side view of an exemplary vape pen of the prior art;

FIG. 2 is a perspective view of a testing device in accordance with an exemplary embodiment of the present invention;

FIG. 3 is a perspective view of a radial fan of the device of FIG. 2, shown with a cover removed for illustrative clarity;

FIG. 4 is a partial perspective view of the device of FIG. 3, showing a vaporizer actuator of the device in greater detail for illustrative clarity;

FIG. 5 is a partial perspective view of the device of FIG. 3, showing a control system compartment of the device in greater detail for illustrative clarity;

FIG. 6 is a partial rear perspective view of the device of FIG. 3, showing a vaporizer holder of the device in greater detail for illustrative clarity;

FIG. 7 is a perspective view of a lower portion of the sample exposure chamber of the device of FIG. 3;

FIG. 8 is a perspective view of an upper portion of the sample exposure chamber of the device of FIG. 3;

FIG. 9 is a perspective view of the upper and lower portions of the sample exposure chamber of the device of FIG. 3, shown open and with vapor flowing for illustrative clarity;

FIG. 10 is a perspective exploded view of the device of FIG. 3;

FIGS. 11-15 are views of the upper and lower portions of the exposure chamber of the device of FIG. 3;

FIGS. 16 and 17 are front and rear perspective views, respectively, of a radial fan of the device of FIG. 3;

FIGS. 18 and 19 are front and rear perspective views, respectively, of a motor cap of the device of FIG. 3;

FIGS. 20 and 21 are front and rear perspective views, respectively of a fan base of the device of FIG. 3;

FIGS. 22 and 23 are front and rear perspective views, respectively, of a fan cover of the device of FIG. 3;

FIGS. 24 and 25 are front and rear perspective views, respectively, of a vaporizer mount of the device of FIG. 3;

FIGS. 26 and 27 are front and rear perspective views, respectively, of the device of FIG. 3, showing vapor flow paths through the device for illustrative clarity;

FIG. 28 is a front perspective view of the device of FIG. 3, showing the vaporizer actuator in an operative position in contact with a button of a vape pen;

FIG. 29 is a front perspective view of the device of FIG. 3, showing the vaporizer actuator in an inoperative position in which it is not in contact with a button of a vape pen;

FIGS. 30A and 30B are diagrammatic views of a vaporizing experiment involving exposure of human airway tissue to experimental vapors using the device of FIG. 3;

FIGS. 31A-34C illustrate experimental results from vaporizing experiments involving exposure of human airway tissue to experimental vapors using the device of FIG. 3;

FIG. 35 is a side view of an alternative embodiment of an alternative testing device in accordance with another exemplary embodiment of the present invention; and

FIG. 36 is an exploded side view of the room air monitoring device of FIG. 35.

### DETAILED DESCRIPTION

The present invention provides a testing device for testing the impact of vaporizable fluids on human airway tissue using a human airway model so that the safety (or risks) of vaping and/or particular vape liquids can be assessed. The device is sufficiently lightweight and compact to be readily usable within a conventional fume hood, so that device-specific ventilation and/or fume control structure and associated cost and complexity can be avoided, to the safety (or risks) of vaping and/or particular vape liquids can be assessed.

The air-liquid interface (ALI) human airway model system has existed for over 20 years and is used in various lines of research to generate functioning airway mucociliary tissue in-vitro from adult stem cells. ALI tissue can be maintained for months in culture, allowing for investigations in airway tissue responses after exposure to experimental compounds of interest. Currently, investigators looking to study the effects that known or experimental compounds have on ALI tissues have minimal options for standardized exposure systems. Researchers that use the ALI model system to investigate effects of topics such as cigarette smoke or electronic cigarette vapors will construct their own devices or use large and expensive laboratory smoking machines that have been retrofitted for ALI tissues.

The present invention provides a compact and automated device that can be used to hold ALI tissue within an exposure chamber while fans create a flow of air through the chamber. The device can deliver aerosolized compounds to ALI tissues from a variety of sources including but not limited to cigarettes, electronic cigarettes, metered-dose inhalers, and nebulizers. The present invention thereby provides a low-cost, compact alternative to a limited selection of bulky and expensive lab equipment, offering an animal-free system to ethically expand the research design and feasibility of human airway disease and toxicology investigations.

In an exemplary embodiment, the housing and fans of the device are 3-D printed from a biocompatible photopolymer resin, e.g., using a FormLabs3b dental 3D-printer. In a preferred embodiment, the housing is formed of transparent material, such as Dental LT Clear (V2).

In a certain embodiment, the fan is driven by a 6-volt miniature electric motor that is controlled by an Arduino nano microcontroller. In certain embodiments, the device may be powered via a standard 120-volt power outlet.

In an exemplary embodiment, the fan is a radial fan, and thus the device employs a radial fan-driven air blower to create the air flow into the exposure chamber instead of syringes or air compressors, which allows for a particularly compact and lightweight device as compared with prior art devices. Accordingly, the device is smaller than free-standing smoking research machines, which makes it fit easily into chemical fume hoods and biosafety cabinets, and to avoid associated device cost and complexity. Further, the components for the device may be 3D printed, which makes production less demanding. Further still, in a preferred embodiment, the device is made from durable biocompatible materials that are autoclavable, which allows for simple and effective sterilization between experiments.

Combining the device with the ALI model system provides an alternative option to expensive animal models for the investigator studying the effects that various aerosolized compounds have on respiratory tissues. For the investigators already utilizing ALI tissue, the device is an affordable alternative to expensive research equipment. There are few international companies that produce cigarette smoking research equipment which is expensive, bulky, and retrofitted for ALI tissues, also there are very limited options with these devices for studies outside of cigarette/electronic cigarette studies. The device is a compact and affordable laboratory device that is designed specifically to be paired with the ALI tissue model system to simplify and standardize a range of aerosol exposure applications. Advantageously, the device is much more compact, and the exposure chamber and air flow generator are combined into a single integrated unit for simplicity and ease of transport.

The device is a compact, all-in-one system for delivering experimental aerosols to the surface of cultured air-liquid interface tissues or other cultured cells. This is accomplished by a series of fan-driven air-intakes which can draw in air from a variety of sources including but not limited to: electronic cigarettes, corticosteroid inhalers, and nebulizers. Accordingly, for example, the device may be used for basic research into the mechanisms of respiratory inflammation, drug delivery, inhalation toxicity, and modelling of inflammatory respiratory diseases, as well as for translational research and personalized medicine using patient tissues for drug/inflammatory studies.

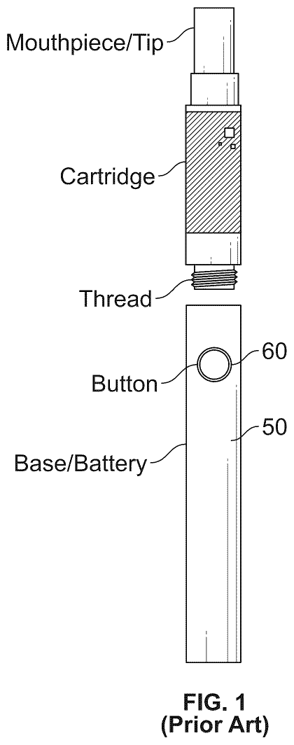

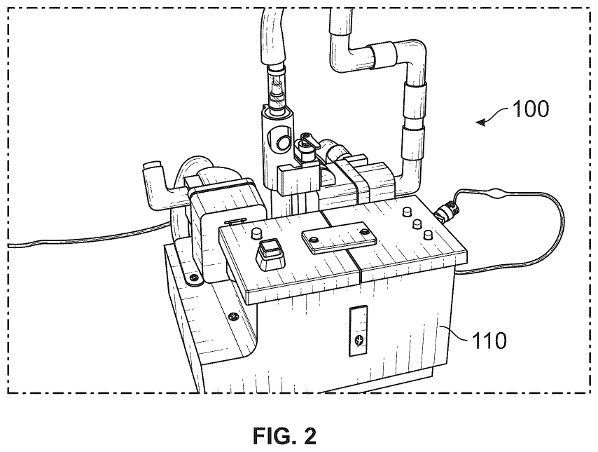

FIG. 2 is a perspective view of an exemplary testing device 100 in accordance with an exemplary embodiment of the present invention. The exemplary device 100 comprises a housing 110 including an upper exposure chamber housing 120 matable with a lower exposure chamber housing 130, as will be appreciated from FIGS. 1, 10, 11 and 12. The upper exposure chamber housing 120 is matable with the lower exposure chamber housing 130 to define an airtight or otherwise substantially closed exposure chamber for exposing tissue samples to vaporized substances.

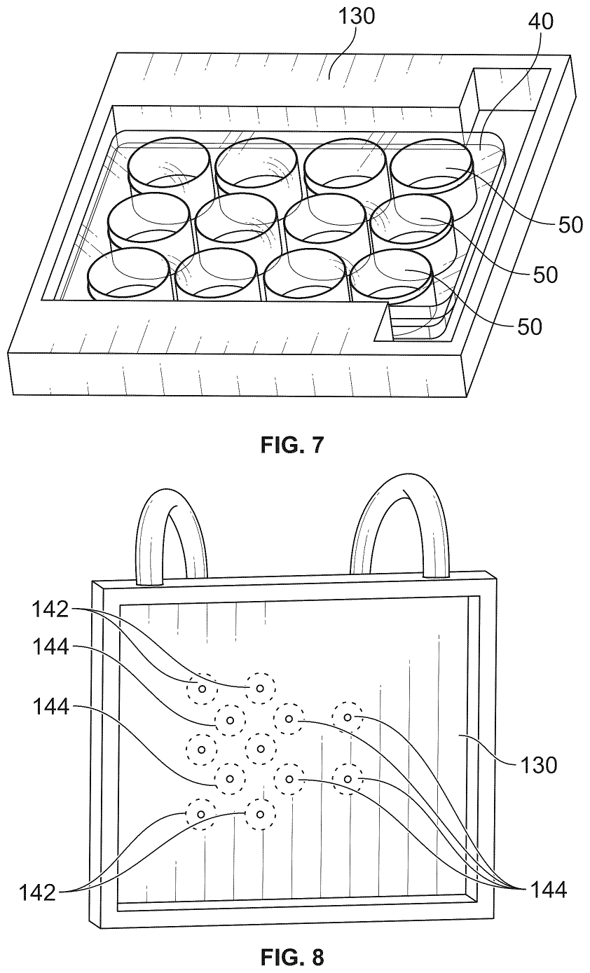

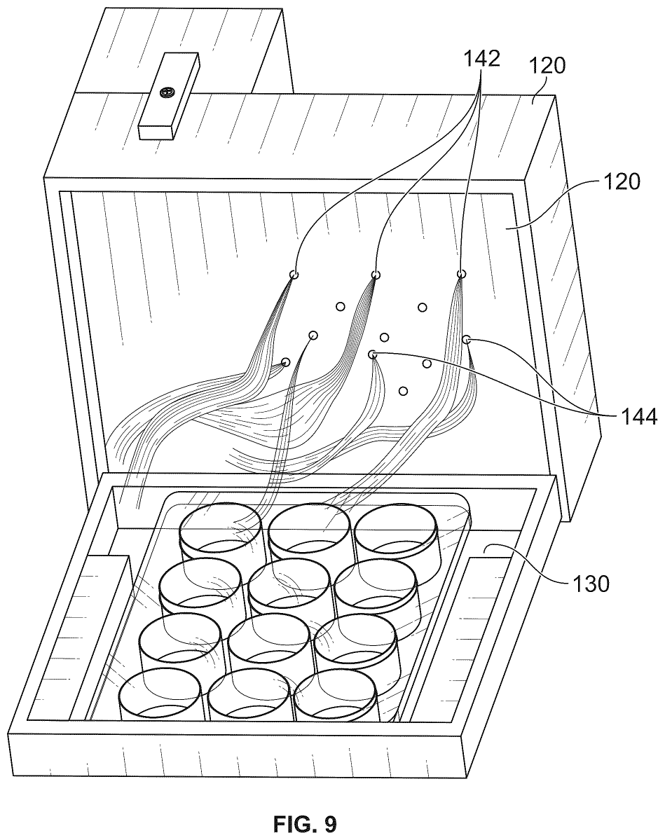

The lower exposure chamber housing 130 has a bottom wall 132 and side walls 134 that collective define an internal chamber 138 for receiving a well plate 40 capable of receiving wells 50, such as a 12-well culture plate, each holding tissue samples, as will be appreciated from FIGS. 7, 9 and 10. In this exemplary embodiment, the exposure chamber fits a well-plate, in this case a 12-well plate, with very close dimensional tolerances, and the exposure chamber is dimensioned to have a T-shape at one end allows additional space and clearance, outside the dimensions of the well plate, for the operator's fingers to fit inside the exposure chamber to grab the well plate when inserting or removing it from the exposure chamber.

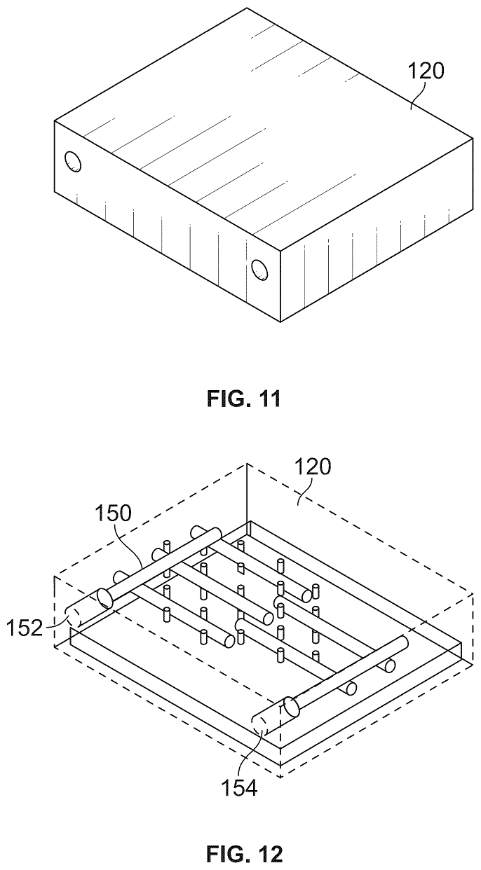

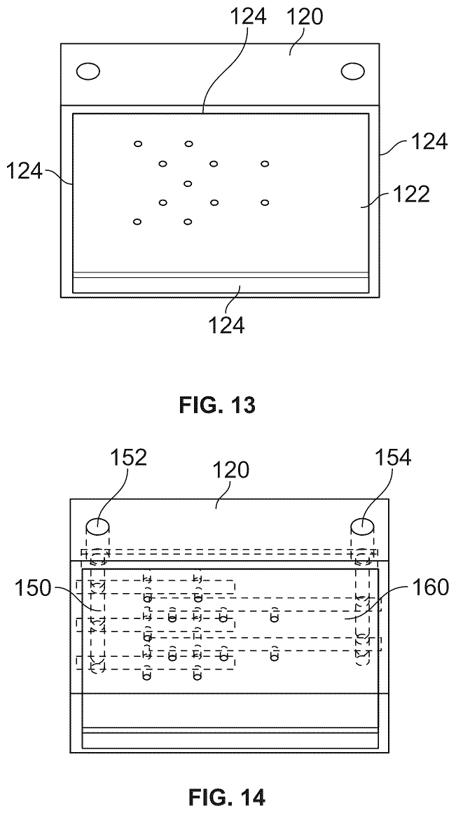

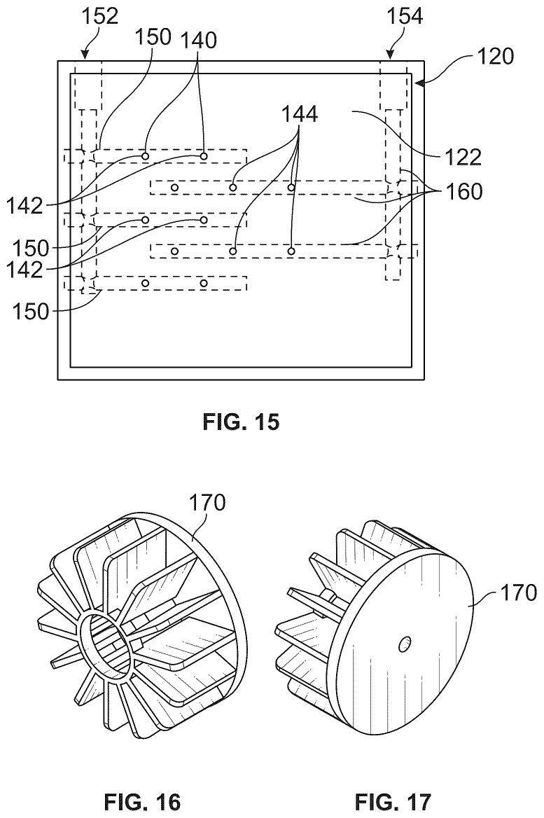

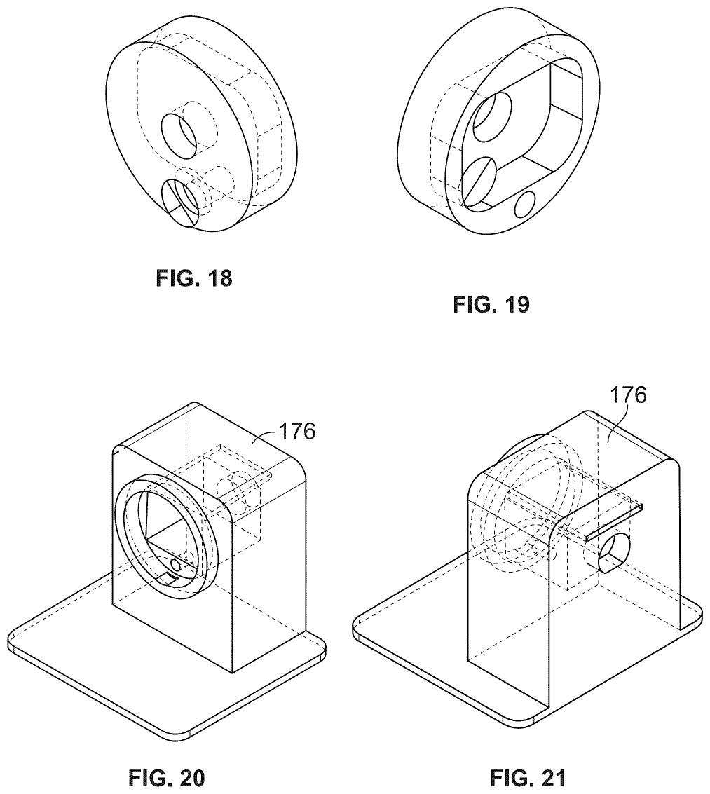

Referring now to FIGS. 1, 7-10, and 11-15, the upper exposure chamber housing 120 has a top wall 122 and side walls 124. The upper exposure chamber housing 120 defines openings 140 acting as intake openings 142 for admitting vapor into the exposure chamber, and exhaust openings 144 for exhausting vapor from the exposure chamber, as will be best appreciated from FIGS. 8, 9 and 10. Further, internal structures of the upper exposure chamber housing 120 define inlet manifold internal passages 150 between the intake openings 142 and intake port 152 in fluid communication with an intake fan for receiving a flow of vapor and directing it into the exposure chamber, and dispersing it via the intake openings 142. Further still, internal structures of the upper exposure chamber housing 120 define exhaust manifold internal passages 160 between the exhaust openings 144 and exhaust port 154 in fluid communication with an exhaust fan for creating vacuum pressure to exhaust vapor from the exposure chamber, via the exhaust openings 144.

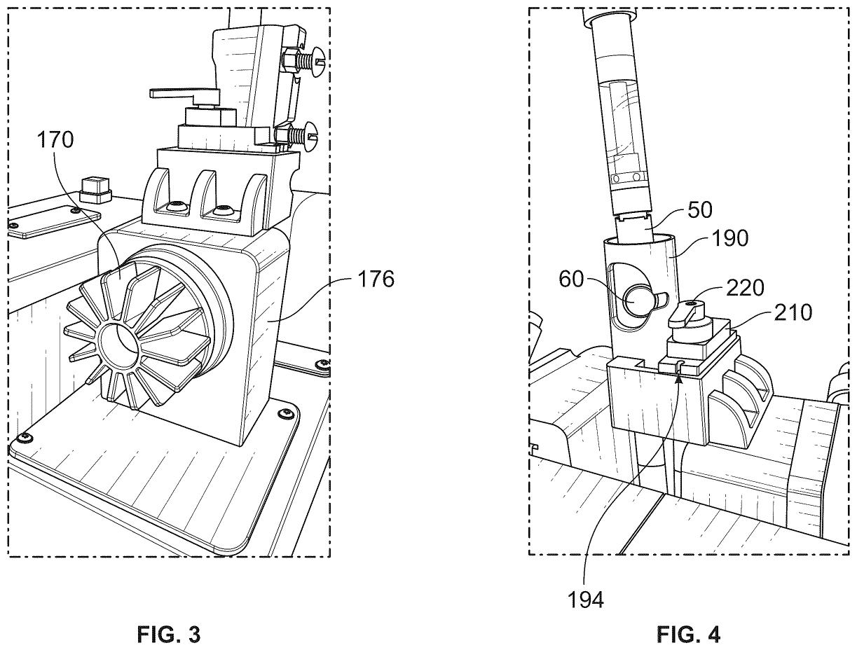

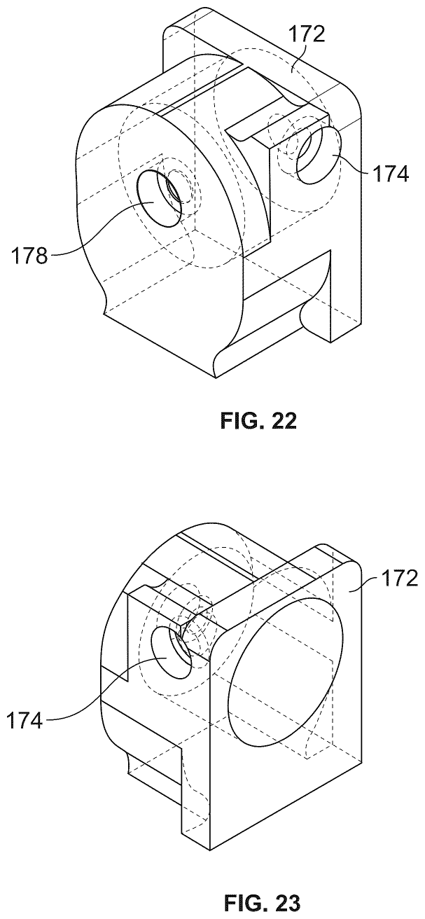

FIGS. 3, 16 and 17 show perspective views of an intake radial fan 170, with an intake cover 172 removed. The intake cover is best shown in FIGS. 1 and 10, 22 and 23. The intake fan 170 and intake cover 172 may be supported on a fan base 176 configured to support an electric motor to which the fan 170 is attached and/or the fan cover 172, as will be appreciated from FIGS. 1, 10, 20 and 21. The intake cover 172 defines an inlet port 178 for receiving an aerosolized compound from the vape pen, etc., and an outlet port 174 connectable (e.g., via tubing, as shown in FIG. 27) to the intake port 152 of the upper exposure chamber housing, as will be appreciated from FIGS. 1, 10, and 27. The intake fan 170 is selectively driven to provide a flow of air/vapor into the exposure chamber, e.g. from the vape pen as shown in FIG. 1. The radial fan configuration is particularly compact and thus desirable in the current configuration, although alternative fan configurations may be used.

A similar radial fan is provided as an exhaust radial fan 180, and exhaust cover, as will be appreciated from FIGS. 1, 10, 16, 17, 22 and 23. The exhaust fan 180 and exhaust cover 182 may be supported on a fan base 186 configured to support an electric motor to which the fan 180 is attached and/or the fan cover 182, as will be appreciated from FIGS. 1, 10, 20 and 21. The exhaust cover 182 defines an inlet port 188 connectable (e.g., via tubing, as shown in FIG. 27) to the exhaust port 154 of the upper exposure chamber housing for drawing the aerosolized compound from the exposure chamber, and an outlet port 184 for exhausting the vented aerosolized compound from the exposure chamber, as will be appreciated from FIGS. 1, 10, and 27. Optionally, the outlet portion 184 may be connected to tubing with an opening venting to the atmosphere, e.g., when the device will be used within a conventional fume hood. The exhaust fan 180 is driven to provide a flow of air/vapor into the exposure chamber, e.g., from the vape pen as shown in FIG. 1. The exhaust fan 180 is selectively driven to provide a flow of air/vapor into the exposure chamber, e.g., from the vape pen as shown in FIG. 1. The radial fan configuration is particularly compact and thus desirable in the current configuration, although alternative fan configurations may be used.

In this embodiment, the exemplary device is adapted for use with a vape pen for generating a vapor. Accordingly, the exemplary device includes a vape pen holder 190 supported on the housing of the device, as shown in FIGS. 1, 2, 4, 6, 10, 24 and 25. The vape pen holder 190 is configured to support the vape pen 50 with its vapor-generating button 60 exposed, and in a suitable position for attachment to intake tubing of the device and in a predetermined position in relation to an actuator for operating the vape pen holder, as best shown in FIGS. 1 and 4. In this manner, the device makes use of a relatively inexpensive vape pen 50 to generate the vapor, and does not require or replicate vapor-generating equipment and/or controls in the testing device, but rather leverages an existing, commercially-available vape pen. The exemplary vape pen holder further includes adjustable set screws 192 adjustable to secure a vape pen in the vape pen holder 190, as shown in FIGS. 6 and 25.

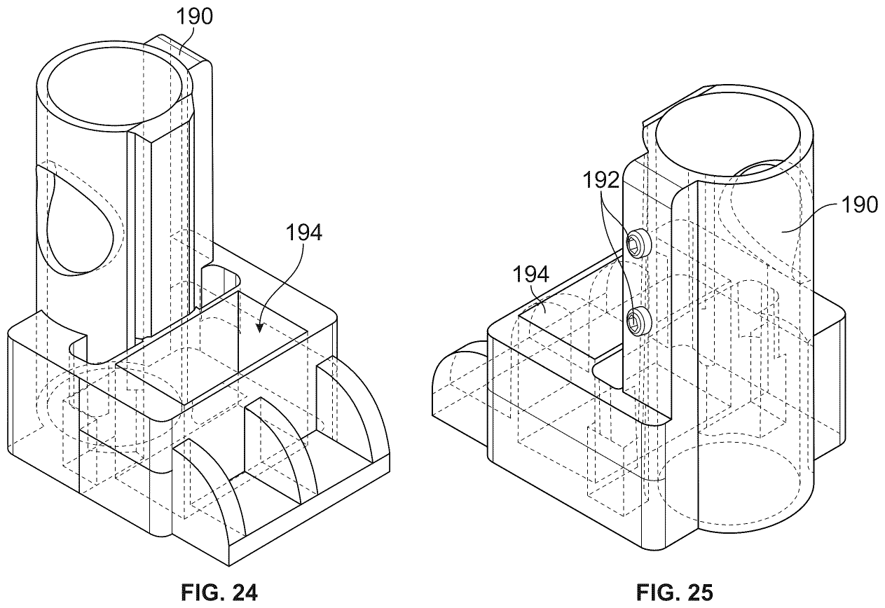

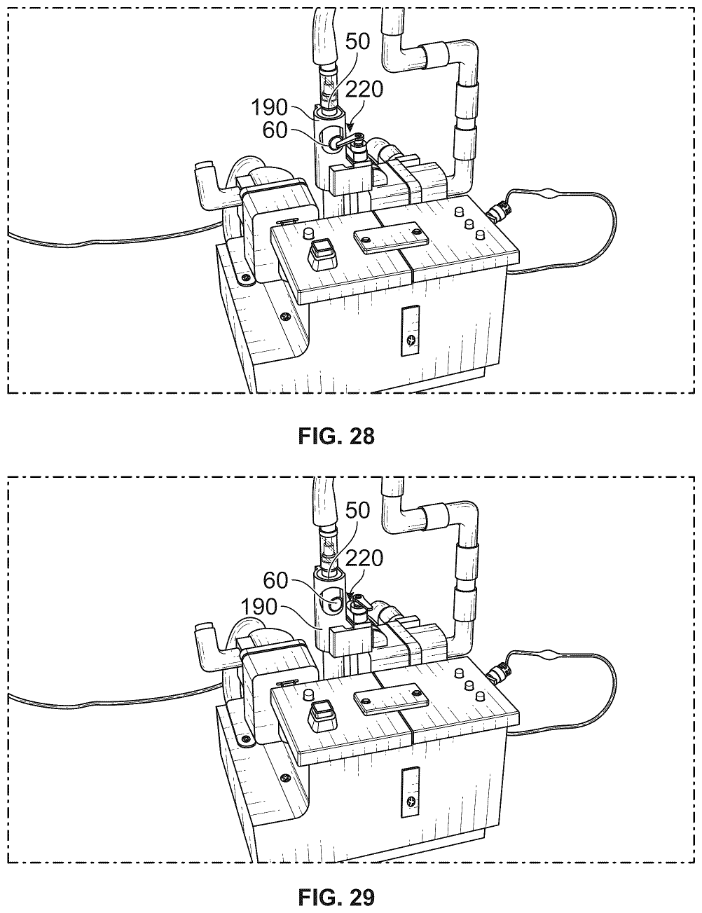

The actuator 200 includes a micro servo 210 having an arm 220. controlled by a control system 250. The micro servo 210 and/or arm 220 is mounted on the housing of the device in a position such that the arm 220 is pivotable between an operative position (in which the arm 220 is in engagement with and depressing a button of a vape pen positioned in the vape pen holder 190, as shown in FIG. 28) and an inoperative position (in which the arm 220 is not in engagement with the button of the vape pen positioned in the vape pen holder 190, as shown in FIGS. 4 and 26, 27 and 29). Optionally, the vape pen holder 190 may be configured to define a socket 194 for receiving and retaining the micro servo 210 in a desired orientation relative to the vape pen holder 190 and actuator/arm, as best shown in FIGS. 4 and 24.

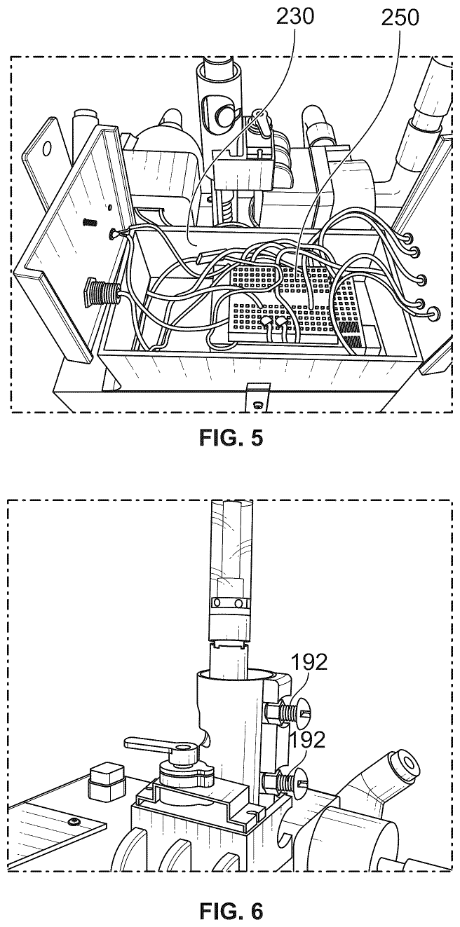

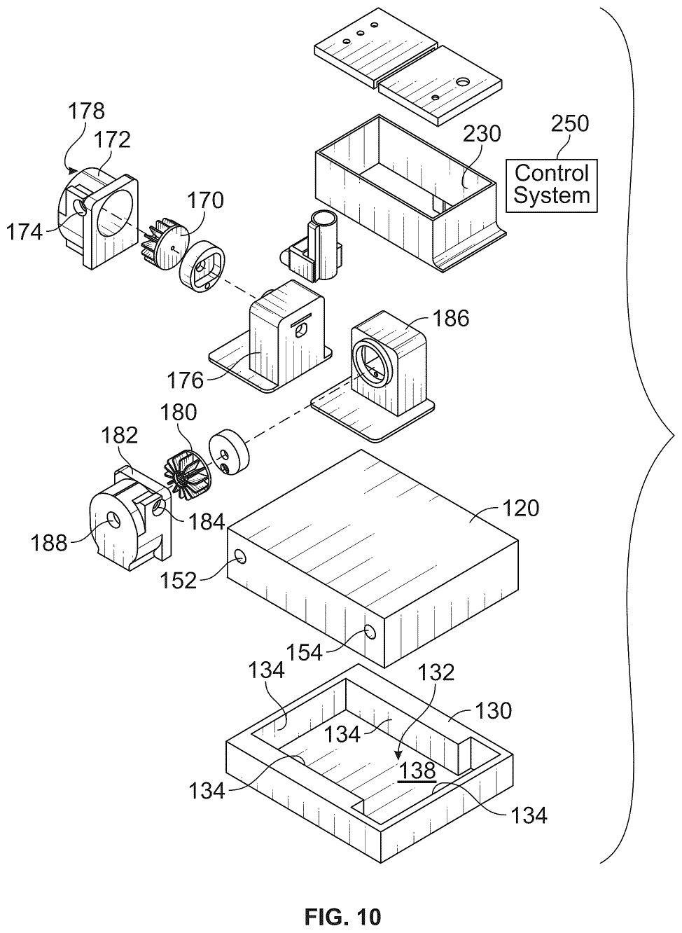

The housing of the device further defines a control system compartment 230, as shown in FIGS. 5 and 10. The compartment 230 receives and houses the control system 250, as shown in FIGS. 5 and 10. In this exemplary embodiment, the control system 250 includes a commercially available Arduino microcontroller. In accordance with the present invention, the microcontroller is configured in accordance with the present invention to operate the micro servo 210/arm 220, intake fan 170 and exhaust fan 180 selectively in accordance with the objectives of the present invention.

In use, multiple wells loaded with ALI tissue may be placed in the lower housing of the exposure chamber, and the upper housing may be mated to the lower housing to provide a closed/substantially closed exposure chamber, as will be appreciated from FIG. 7.

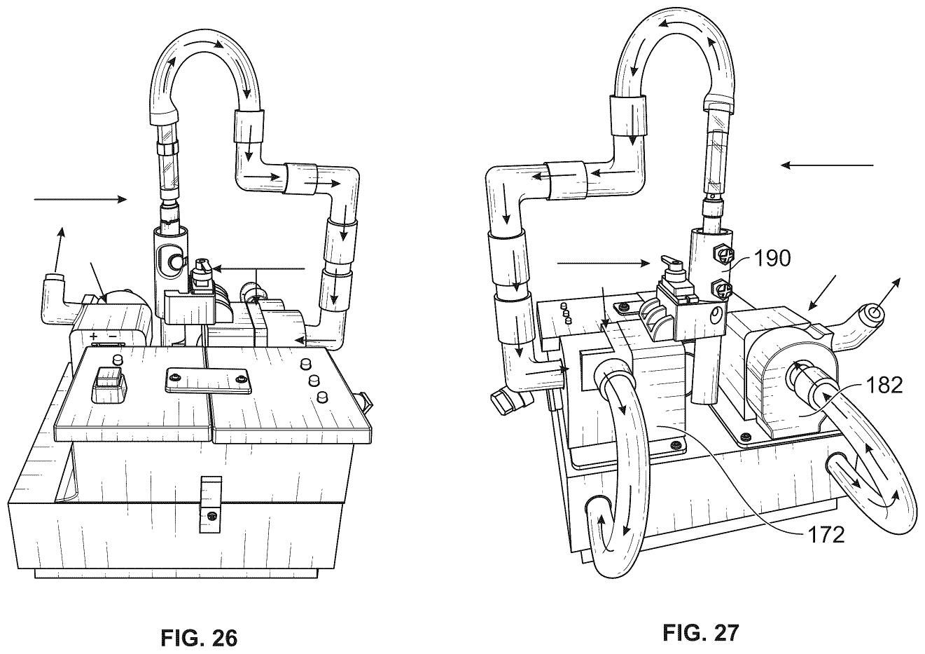

Referring now to FIGS. 26 and 27, a vape pen loaded with a cartridge filled with vape fluid to be tested may be loaded into a convention vape pen. The vape pen may then be positioned in the vape pen mount with its button positioned to be selective engaged with the arm/actuator of the micro servo. The set screws may be advanced to secure the vape pen in the vape pen holder. Intake tubing may be connected at one end to the mouthpiece and at the other end to an intel port on the intake fan housing to place the vape pen in fluid communication with the intake fan.

Intake tubing is provided to place the exhaust port of the intake fan housing in fluid communication with the intake port of the exposure chamber/upper housing. Similarly, exhaust tubing is provided to place the intake port of the exhaust fan housing in fluid communication with exhaust port of the exposure chamber/upper housing. The exhaust port of the exhaust fan housing may exhaust to the atmosphere in embodiments in which the device is intended to be used under an existing fume hood, which is relied upon for proper handling of the flow of exhaust from the device.

During operation, the control system may then energize the micro servo to cause the actuator/arm to rotate into the operative position in which it is abutting and depressing the button of the vape pen, as shown in FIG. 28. This causes the vape pen to vaporize the vape fluid in the cartridge and to make the vapor available for withdrawal via the mouthpiece of the vape pen.

The control system may then cause the intake fan to be energized to create a vacuum and/or draw a flow of air into/through the exposure chamber. As a result, the vapor is drawn through the mouthpiece, through the intake tubing and through the intake fan housing and into the exposure chamber, and a result of operation of the intake fan. This exposes the tissues in the wells in the exposure chamber to the vapor.

After a period of time, the control system may then energize the micro servo to cause the actuator/arm to rotate into the inoperative position in which it is no longer abutting and depressing the button of the vape pen, as shown in FIG. 29. This causes the vape pen to stop vaporizing the vape fluid in the cartridge.

After a period of time, the control system may then stop the intake fan from being energized to stop the vacuum and/or flow of air through the exposure chamber. The control system may then cause the exhaust fan to be energized to create a vacuum and/or draw a flow of air out of the exposure chamber. As a result, the vapor is drawn from the exposure chamber, through the exhaust tubing and through the exhaust fan housing and expelled from the device, and a result of operation of the exhaust fan. This clears the exposure chamber of vapor.

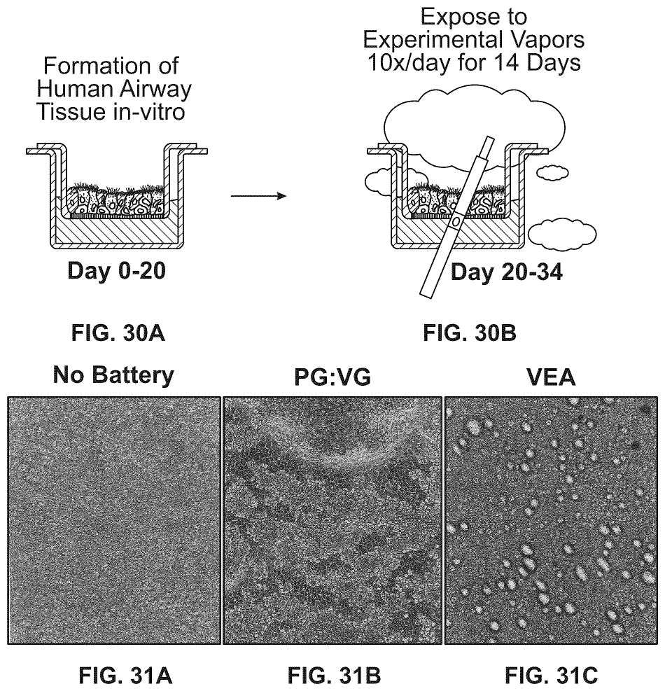

Referring now to FIGS. 30A-30B, 31A-31C, 32, 33A-33C, 34A-34C, e-cigarette research investigation conducted using the device. The investigation compared how human airway model tissue was affected by e-cigarette vapors generated from two different vape-liquids, propylene glycol/vegetable glycerin ‘PG:VG’, and vitamin E acetate ‘VEA’.

Referring to FIGS. 30A-30B, illustration of experimental design used in research investigation, human airway model tissues were formed from day 0 to day 20, tissues were exposed to experimental vapors from day 20 to day 34.

Referring to FIGS. 31A-31C, phase contrast microscopy images of the surfaces of live human airway model tissues after being exposed to experimental vapors. The untreated ‘no battery’ tissue appears typical, Propylene glycol/vegetable glycerin ‘PG:VG’ tissues appear damaged, vitamin E acetate ‘VEA’ tissues have oil droplets on the surface, but the tissue appears un-damaged.

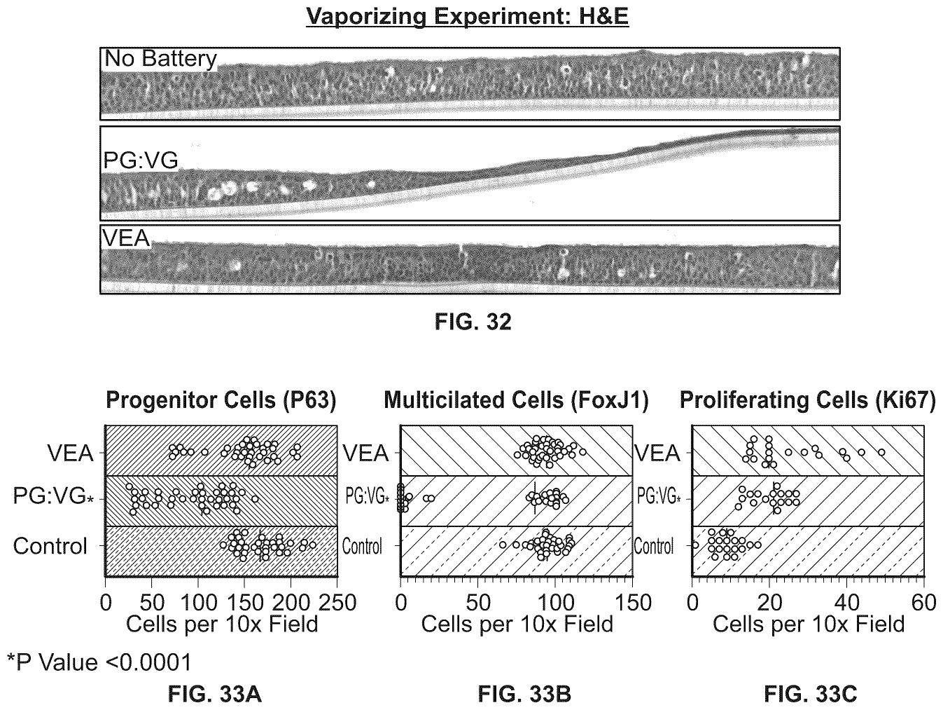

Referring to FIG. 32, brightfield microscopy images of cross-sections of experimental tissues stained with specialty stain to visualize tissue morphology, the untreated ‘no battery’ and the vitamin e acetate ‘VEA’ tissues present typical structure and are undamaged, the propylene glycol/vegetable glycerin ‘PG:VG’ tissue is damaged as evident by reduced thickness of tissue.

Referring to FIGS. 33A-33C, dot plot graphs representing cell-type populations within experimental tissues, cell counts were collected manually from fluorescent microscope images of tissue sections stained with cell-specific markers. Progenitor cell populations are reduced, and multiciliated cells are absent in areas of tissues exposed to propylene glycol/vegetable glycerin ‘PG:VG’ vapors, indicating increased cell death rate due to vapor toxicity. The study also observed increase in proliferating cells in tissues exposed to either of the experimental vapors, indicating increased cell-cycle activation due to increased cell death.

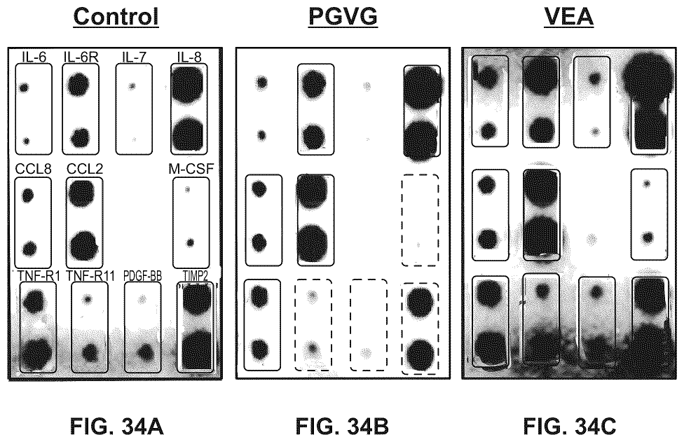

Referring to FIG. 34A-34C, commercially available human inflammation cytokine array performed on mucosal washes collected from the surfaces of experimental tissues. Multiple inflammatory targets are increased in mucosal washes collected from the surfaces of tissues exposed to experimental vapors compared to untreated ‘control’ tissues, indicating activation of multiple inflammatory pathways due to experimental vapor exposure.

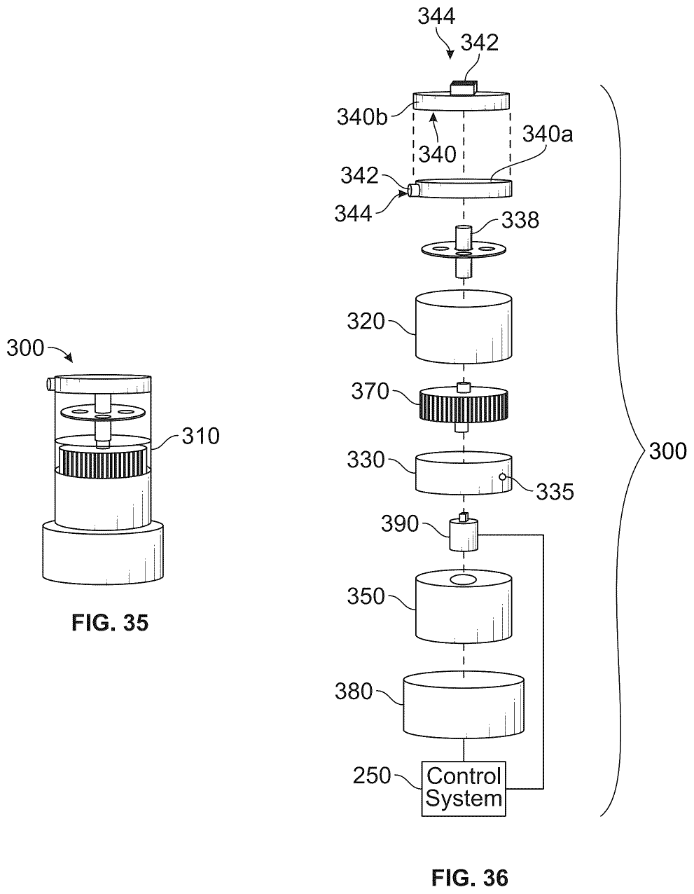

Referring now to FIGS. 35 and 36, and alternative embodiment of the testing device is shown. This exemplary testing 300 is a compact, all-in-one system that uses a fan-driven air-intake to draw vapor through the device, through an exposure chamber that houses the ALI tissue sample, and out of the device and housing 310 via an exhaust port 335.

Referring now to FIGS. 35 and 36, the exemplary testing device 300 has a compact, upright form factor and comprises a housing 310 including an exposure chamber housing 320 that is matable with a chamber cap 340 and a fan housing 330. Together with the chamber cap 340 and the fan housing 330, the exposure chamber housing 320 defines an internal exposure chamber 338 dimensioned to receive a stand 134 configured to hold wells and/or ALI or other tissue culture, or other, material. In use, the wells with tissue/tissue may be placed into the stand 134, and the stand may be placed in the exposure chamber, as will be appreciated from FIG. 35.

The chamber cap 340 covers the top of the exposure chamber housing to keep out debris, and preferably forms a substantially enclosed exposure chamber. The chamber cap 340 defines one or more openings 342 suitable to its application. In one exemplary embodiment, the chamber cap 340a defines an opening 342 providing a port 344 suitable for connection to tubing that may be attached to a mouthpiece of a vaping device, to admit passage of aerosolized compounds from the vaping device to be supplied to the exposure chamber 338 for testing purposes. In another exemplary embodiment, the chamber cap 340b defines an opening providing a port 348 suitable for mating to an outlet of a metered asthma inhaler or an adapter, to admit passage of aerosolized compounds from the vaping device to be supplied to the exposure chamber 338 for testing purposes.

The fan housing 330 is dimensioned to house an intake fan 370, such as a radial fan. The intake fan 370 is driven to provide a flow of ambient air the exposure chamber, e.g., through the openings 342 of the chamber cap 340, and exiting the device via multiple openings in the bottom of the exposure chamber that lead into the fan housing which has at least one opening that acts as the final exit point of ambient air from the device (not shown). A radial fan configuration is particularly compact and thus desirable in the current configuration, although alternative fan configurations may be used.

The fan housing 330 is configured to sit atop and/or otherwise mate with a fan motor housing 350. The fan motor housing 350 is dimensioned to house and support an electric motor 390 that is mechanically interconnected with the fan 370 to drive rotational motion thereof when the electric motor 390 is energized. The electric motor may be a 6-volt miniature electric motor. The electric motor 190 is further operatively coupled to a control system 400 configured for driving the fan. The control system 400 is configured with logic operative to cause the control system 400 to selectively energize or otherwise control operation of the motor and fan to provide a desired exposure of aerosolized compounds to any sample contained in the exposure chamber, to provide a suitable dwell time, etc.

The fan motor housing 350 is configured to sit atop and/or otherwise mate with a control system housing 380. The control system housing 380 is dimensioned to house and support the control system 400, which may include an Arduino nano or other microcontroller suitably configured for driving the fan 370, a power supply, etc.

During operation, the control system 400 energizes the fan motor 390 and causes it to run, e.g., continuously or for intermittent cycles. This causes the fan 370 to draw air, which may contain aerosolized compounds when the device is mated to a vaping device, asthma inhaler or other similar device via an opening/port of the chamber cap 340, into the exposure chamber 338 and into contact with any ALI tissue or other sample or other material in any wells in the stand 338, and then to exit the device via at least one opening in the bottom of the exposure chamber that leads into the fan housing which has at least one opening that acts as the final exit point of air (not shown). This exposes the tissues or other sample in the exposure chamber to the aerosolized compounds admitted via the port 344.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. An analytical testing device for testing the impact of an aerosolized compounds on human airway tissue, the device comprising:
an intake fan assembly comprising an intake fan and an intake fan electric motor operatively connected to the intake fan to drive rotation of the intake fan when the intake fan electric motor is energized;
an exhaust fan assembly comprising an exhaust fan and an exhaust fan electric motor operatively connected to the exhaust fan to drive rotation of the exhaust fan when the exhaust fan electric motor is energized;
a housing defining a substantially-closed exposure chamber, said housing defining an intake port in fluid communication with said intake fan and inlet openings in fluid communication with said intake port and said exposure chamber, said housing further defining an exhaust port in fluid communication with said exhaust fan and exhaust openings in fluid communication with said exhaust port and said exposure chamber;
a holder supported on said housing and configured to support an aerosol-generating device operable to generate an aerosolized compound in response to actuation of a switch;
an actuator supported on said housing and configured to be movable between an operative position that causes the aerosol-generating device's switch to be in a position that will cause generating of the aerosolized compound, and an inoperative position that causes the aerosol-generating device's switch to be in a position that will not cause generating of the aerosolized compound; and
a control system operable to selectively operate said actuator to cause generation of an aerosolized compound, to selectively energize said intake fan electric motor to cause said intake fan to draw the aerosolized compound into said exposure chamber, and to selectively energize said exhaust fan electric motor to cause said exhaust fan assembly to exhaust the aerosolized compound from said exposure chamber.

2. The analytical testing device of claim 1, wherein each of said intake fan and said exhaust fan comprises a respective radial fan.

3. The analytical testing device of claim 1, wherein said housing, said intake fan and said exhaust fan are constructed of a biocompatible material that is autoclavable without detrimental material degradation.

4. The analytical testing device of claim 3, wherein said biocompatible material is a photopolymer resin.

5. The analytical testing device of claim 3, wherein said housing, said intake fan and said exhaust fan are constructed using an additive manufacturing process.

6. The analytical testing device of claim 1, wherein said housing comprises:
a lower exposure chamber housing defining a cavity dimension to receive a multi-well plate; and
an upper exposure chamber housing dimensioned to be matable with the lower exposure chamber housing to define said exposure chamber.

7. The analytical testing device of claim 6, wherein said upper exposure chamber housing has a top wall and side walls, and wherein said top wall defines said intake openings and said exhaust openings.

8. The analytical testing device of claim 7, wherein said upper exposure chamber housing defines inlet manifold internal passages between said intake openings and said intake port.

9. The analytical testing device of claim 7, wherein each of said intake openings is positioned to be vertically aligned with a well of a well plate positioned within said lower exposure chamber housing.

10. The analytical testing device of claim 7, wherein said upper exposure chamber housing defines exhaust manifold internal passages between said exhaust openings and said exhaust port.

11. The analytical testing device of claim 1, wherein said aerosol-generating device is a vape pen, and wherein said holder is configured to receive and support said vape pen in a defined position relative to said actuator.

12. The analytical testing device of claim 1, wherein said aerosol-generating device is one of an electronic cigarette, a vape pen, an inhaler, and a nebulizer, and wherein said holder is configured to receive and support said aerosol-generating device in a defined position relative to said actuator.

13. The analytical testing device of claim 1, wherein said actuator comprises a micro servo having an arm controlled by said control system.

14. The analytical testing device of claim 1, wherein said housing further defines a control system compartment dimensioned to receive and house the control system in a substantially-enclosed fashion.

15. The analytical testing device of claim 1, wherein said control system comprises a microcontroller.

16. The analytical testing device of claim 1, wherein said control system is configured with predetermined logic for timing of operation of said actuator, said intake fan motor, and said exhaust fan motor.

17. The analytical testing device of claim 16, wherein said control system is configured with predetermined logic to allow for passage of a predetermined period of time between energizing of said intake fan motor and energizing of said exhaust fan motor to provide a prescribed dwell time of said aerosolized compound in said exposure chamber.

18. A method for testing the impact of an aerosolized compounds on human airway tissue, the method comprising:
providing an analytical testing device for testing the impact of an aerosolized compound on human airway tissue, the device comprising:
an intake fan assembly comprising an intake fan and an intake fan electric motor operatively connected to the intake fan to drive rotation of the intake fan when the intake fan electric motor is energized;
an exhaust fan assembly comprising an exhaust fan and an exhaust fan electric motor operatively connected to the exhaust fan to drive rotation of the exhaust fan when the exhaust fan electric motor is energized;
a housing defining a substantially-closed exposure chamber, said housing defining an intake port in fluid communication with said intake fan and inlet openings in fluid communication with said intake port and said exposure chamber, said housing further defining an exhaust port in fluid communication with said exhaust fan and exhaust openings in fluid communication with said exhaust port and said exposure chamber;
a holder supported on said housing and configured to support an aerosol-generating device operable to generate an aerosolized compound in response to actuation of a switch;
an actuator supported on said housing and configured to be movable between an operative position that causes the aerosol-generating device's switch to be in a position that will cause generating of the aerosolized compound, and an inoperative position that causes the aerosol-generating device's switch to be in a position that will not cause generating of the aerosolized compound; and
a control system operable to selectively operate said actuator to cause creation of an aerosolized compound, and to selectively energize said intake fan electric motor to cause said intake fan to draw the aerosolized compound into said exposure chamber, and to selectively energize said exhaust fan electric motor to cause said exhaust fan assembly to exhaust the aerosolized compound from said exposure chamber;
providing human model airway tissue in the exposure chamber of the analytical testing device; and
operating said analytical testing device to pass the aerosolized compound through the exposure chamber of said device, to expose said human model airway tissue to said aerosolized compound.

19. The method of claim 18, further comprising:
analyzing said human model airway tissue exposed to said aerosolized compound to determine an impact of said aerosolized compound on said human model airway tissue.

20. The method of claim 18, wherein said providing human model airway tissue in the exposure chamber of the analytical testing device comprises disposing air-liquid interface human airway model ins the analytical testing device.

21. An analytical testing device for testing the impact of an aerosolized compounds on human airway tissue, the device comprising:
an intake fan assembly comprising an intake fan and an intake fan electric motor operatively connected to the intake fan to drive rotation of the intake fan when the intake fan electric motor is energized;
a housing defining a substantially-closed exposure chamber, said housing defining an intake port in fluid communication with said intake fan and inlet openings in fluid communication with said intake port and said exposure chamber, said housing further defining an exhaust port in fluid communication with said exhaust fan and exhaust openings in fluid communication with said exhaust port and said exposure chamber;
a holder supported on said housing and configured to support an aerosol-generating device operable to generate an aerosolized compound; and
a control system operable to selectively energize said intake fan electric motor to cause said intake fan to draw the aerosolized compound into said exposure chamber.

22. The analytical testing device of claim 21, further comprising:
an actuator supported on said housing and configured to be movable between an operative position that causes the aerosol-generating device to generate the aerosolized compound, and an inoperative position that does not cause the aerosol-generating device to generate the aerosolized compound;
wherein said control system is further operable to selectively operate said actuator to cause generation of the aerosolized compound by the aerosol-generating device.

23. The analytical testing device of claim 22, further comprising:
an exhaust fan assembly comprising an exhaust fan and an exhaust fan electric motor operatively connected to the exhaust fan to drive rotation of the exhaust fan when the exhaust fan electric motor is energized;
wherein said control system is further operable to selectively energize said exhaust fan electric motor to cause said exhaust fan assembly to exhaust the aerosolized compound from said exposure chamber.

24. The analytical testing device of claim 21, further comprising:
an exhaust fan assembly comprising an exhaust fan and an exhaust fan electric motor operatively connected to the exhaust fan to drive rotation of the exhaust fan when the exhaust fan electric motor is energized;
wherein said control system is further operable to selectively energize said exhaust fan electric motor to cause said exhaust fan assembly to exhaust the aerosolized compound from said exposure chamber.
